(19) United States
(12) Patent Application Publication
(10) Pub. No.: US 2019/0352329 A1
(43) Pub. Date: Nov. 21, 2019

(54) BETA-ADRENERGIC BLOCKING COMPOUND

(71) Applicant: KING SAUD UNIVERSITY, RIYADH (SA)

(72) Inventors: ABD EL-GALIL E. AMR, RIYADH (SA); MOHAMED A. AL-OMAR, RIYADH (SA); HAZEM AHMED GHABBOUR, RIYADH (SA)

(21) Appl. No.: 15/980,670

(22) Filed: May 15, 2018

(51) Int. Cl. C07J 63/00

(52) U.S. Cl. CPC C07J 63/008

(57) ABSTRACT

A beta-adrenergic blocking compound according to the present teachings include compounds having the following structural formula:

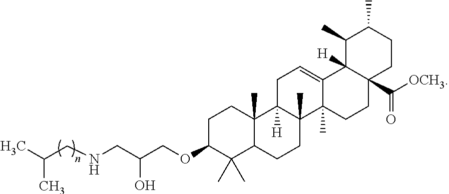

wherein n is 0 or 1,
or a pharmaceutically acceptable salt thereof.

### BACKGROUND

### 1. Field

The disclosure of the present patent application relates to beta-adrenergic blocking compounds, and particularly, to methyl 3β-substituted urs-12-en-28-oate derivatives as potent selective β1-blockers.

### 2. Description of the Related Art

Beta-adrenergic blockers or 3-blockers are frequently used to treat several cardiovascular disorders such as hypertension, cardiac arrhythmia, angina pectoris and open angle glaucoma. Increased systolic and diastolic blood pressure can induce hypertension, which in turn damage the renal, cardiac, and brain blood vessels. Beta-blockers block the action of the sympathetic nervous system of the heart, thus reducing stress on the heart. Beta-blockers block beta-adrenergic substances such as epinephrine (adrenaline) in the autonomic nervous system. They control increase in blood pressure and, thus, inhibit damage to blood vessels. Atenolol is a selective β₁-receptor antagonist, a drug belonging to the group of beta blockers (β-blockers), a class of drugs used primarily in cardiovascular diseases. Atenolol is effective at reducing blood pressure.

Ursolic acid has been shown to inhibit JNK expression and IL-2 activation of JURKAT leukemic T Cells, which leads to reduction in proliferation and T cell activation. Ursolic acid has been shown to stimulate muscular growth in mice and shows potential cardio-protection. Ursolic acid also leads to decreased production of MMP-2 and u-PA. In addition, peroxisome proliferator-activated receptor (PPAR) activation suppressed the inflammatory response in brain ischemia. This finding suggests that PPARy might be critical in cases where the brain is restricted of blood (ischemia). PPARy protein levels were increased after ursolic acid treatment in a dose-dependent manner. Oleanolic and ursolic acid (triterpenoids) prevented the development of severe hypertension, and did not have direct hypotensive effect, after 6-week application with a daily dose of 60 mg/kg b.w., i.p. The antihypertensive effect was attributed to the potent diuretic-natriuretic-saluretic activity; direct cardiac effect (heart rate decrease by 34% and 32%, respectively).

### SUMMARY

A beta-adrenergic blocking compound according to the present teachings includes a compound having the following structural formula:

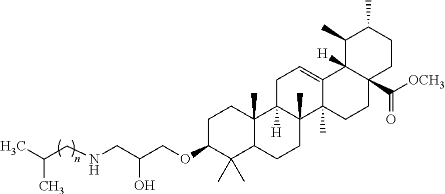

wherein n is 0 or 1,
or a pharmaceutically acceptable salt thereof.

These and other features of the present disclosure will become readily apparent upon further review of the following specification and drawings.

### BRIEF DESCRIPTION OF THE DRAWINGS

FIG. 1 is reaction scheme for synthesis of compounds 3a and 3b according to the present teachings.

FIG. 2 is a graph showing IC₅₀ of the beta blocker activity for compounds 3a and 3b.

FIG. 3A shows binding mode of compound 3a into the binding site of β1-Adrenergic receptor (PDB 2vt4).

FIG. 3B shows binding mode of compound 3b into the binding site of β1-Adrenergic receptor (PDB 2vt4).

Similar reference characters denote corresponding features consistently throughout the attached drawings.

### DETAILED DESCRIPTION OF THE PREFERRED EMBODIMENTS

A beta-adrenergic blocking compound according to the present teachings includes a compound having the following structural formula:

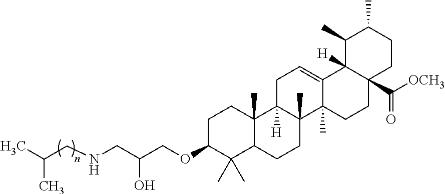

wherein n is 0 or 1,
or a pharmaceutically acceptable salt thereof.

A pharmaceutically acceptable salt includes any non-toxic salt of the present compounds, which are generally prepared by reacting the free acid with a suitable organic or inorganic base. Examples of such salts include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, triethiodide, valerate.

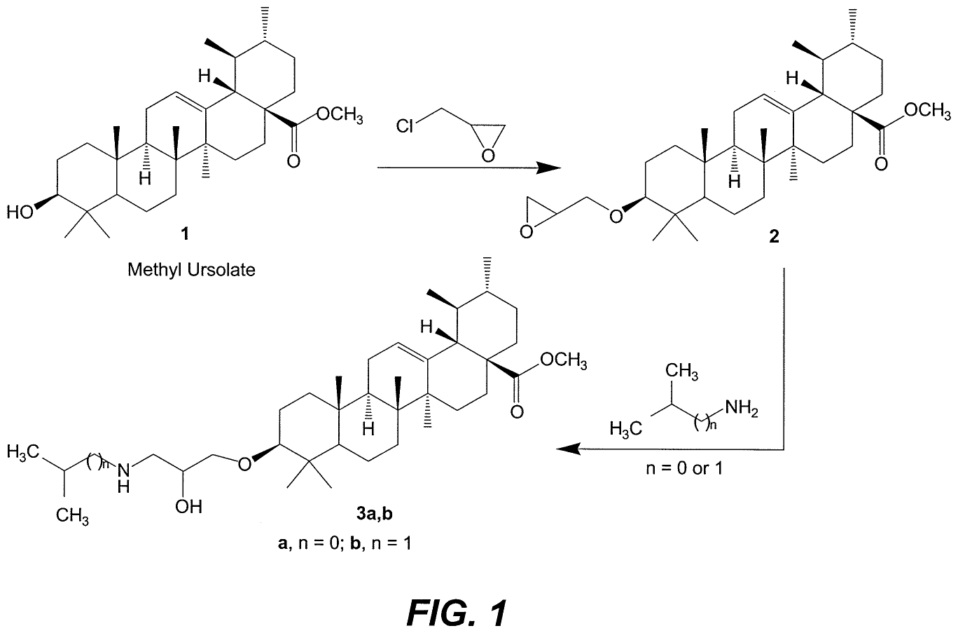

FIG. 1 depicts a reaction scheme by which the exemplary beta-adrenergic blocking compounds can be prepared. With reference to FIG. 1, a method of preparing the present compounds can include condensation of methyl ursolate (1) with 2-(chloromethyl)oxirane (epichlorohydrin) to afford the corresponding methyl-3-β-(2,3-epoxypropoxy)-urs-12-en-28-oate (2); treating compound 2 with amine derivatives, namely, isopropyl or isobutylamine to afford the corresponding beta-adrenergic blocking compounds, namely, methyl 3-β-[2-hydroxy-3-(isopropylamino)propoxy]-urs-12-en-28-oate (3a) and methyl-3-β-[2-hydroxy-3-(isobutylamino)propoxy]-urs-12-en-28-oate (3b), respectively.

The present compounds can be effective beta-adrenergic blocking agents or β-blockers. For example, the present compounds can be selective β1-blockers. As described herein, compounds 3a and 3b blocked isoprenaline inotropic response almost fully as demonstrated by the IC50 values. Both compounds 3a and 3b were more potent than Metoprolol and Atenolol. Compound 3a was more active than compound 3b.

The present compounds can be administered to a patient in need thereof. For example, the compounds can be used to treat a patient suffering from hypertension, high blood pressure, and/or cardiovascular diseases. The compounds can be administered by any conventional route of administration, including, but not limited to, intravenous, oral, subcutaneous, intramuscular, intradermal and parenteral. Depending on the route of administration, the compounds can be constituted into any form. For example, forms suitable for oral administration include solid forms, such as pills, gelcaps, tablets, caplets, capsules (each including immediate release, timed release and sustained release formulations), granules, and powders. Forms suitable for oral administration also include liquid forms, such as solutions, syrups, elixirs, emulsions, and suspensions. In addition, forms useful for parenteral administration include sterile solutions, emulsions and suspensions.

Also provided is a pharmaceutical composition including the beta-adrenergic blocking compound. To prepare the pharmaceutical composition, one or more beta-adrenergic blocking compounds or salt thereof, as the active ingredient, is intimately admixed with a pharmaceutical carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents and the like; for solid oral preparations, suitable carriers and additives include starches, sugars, diluents, granulating agents, lubricants, binders, disintegrating agents and the like. For parenteral use, the carrier will usually comprise sterile water, though other ingredients, for example, for purposes such as aiding solubility or for preservation, may be included. Injectable suspensions may also be prepared, in which case appropriate liquid carriers, suspending agents and the like may be employed.

The present compounds are terpeoidal derivatives having β₁ receptor antagonist activity. The present compounds can achieve more potent antihypertensive effects than Atenolol. The present compounds include ursolic acid which possesses antihypertensive properties. Ursolic acid is known to prevent the development of severe hypertension. The antihypertensive effect can be attributed to the potent diuretic-natriuretic-saluretic activity and/or direct cardiac effect (heart rate decrease by 32%).

The following examples illustrate the present teachings.

### EXAMPLES

All melting points are uncorrected and were measured using an electrothermal capillary melting point apparatus. The IR spectra were recorded on a Shimadzu FT-IR 8101 PC infrared spectro-photometer. The 1H NMR spectra were determined with Bruker AM-400 MHz spectrometer. The chemical shifts are expressed on the δ (ppm) scale using TMS as the standard reference. Mass spectra were recorded on Finnigan SSQ operating at 70 ev. Elemental analysis determined on a Perkin Elmer 240 (microanalysis), Microanalysis Center, Cairo University, Cairo, Egypt.

Data are expressed as means±s. e. mean. IC50s for each drug were estimated through non-linear regression analysis using ISI software for a PC computer. Differences between non paired groups were compared by Student's t-test or ANOVA test with the statistical programme Stat works TM; a value of P equal or smaller than 0.05 was taken as the limit of statistical significance.

### Example 1

### Synthesis of methyl-3-β-(2,3-epoxypropoxy)-urs-12-en-28-oate (2)

A solution of methyl ursolate 1 (4.7 g, 10 mmol) and epichlorohydrin (0.78 ml, 10 mmol) and triethylamine (1.4 ml) in dioxane (100 ml) was refluxed for 7 hours, then stand stirred overnight. Then, the reaction mixture was evaporated to dryness and washed with HCl, followed with water and finally crystallized from benzene petroleum ether to give compound 2. Yield 58%, mp. 225-227° C., [α]_{D}²⁵=+119 (c 1, CHCl₃); IR (KBr, cm⁻¹): 2977 (CH, aliphatic), 1746 (ester), 1634 (C═C), 1275 (ether). ¹H NMR (400 MHz, CDCl₃, δ in ppm): 0.88 (d, 1H, CH), 0.92 (s, 3H, CH₃), 0.97 (d, 3H, CH₃), 0.98 (d, H, 1H, CH), 1.00 (s, 3H, CH₃), 1.02 (d, 3H, CH₃), 1.05 (m, 1H, CH), 1.08 (s, 3H, CH₃), 1.22 (m, H, CH), 1.24 (s, 3H, CH₃), 1.27 (s, 3H, CH₃), 1.36 (m, 1H, CH), 1.39 (m, 1H, CH), 1.42 (m, 1H, CH), 1.46 (m, 1H, CH), 1.49 (m, 1H, CH), 1.53 (d, H, 1H, CH), 1.56 (m, 1H, CH), 1.59 (m, 1H, CH), 1.65 (s, 1H, CH), 1.81 (dd, 2H, CH₂), 1.91 (m, 2H, CH₂), 1.95 (m, 1H, CH), 1.98 (m, 1H, CH), 2.02 (m, 1H, CH), 2.14 (t, 1H, CH), 2.33 (t, 1H, CH), 2.48 (t, 1H, CH, propyl-H), 2.54 (d, 2H, CH₂, propyl-H), 2.63 (d, 1H, 1H, CH), 3.44 (dd, 1H, CH), 3.49 (s, 314, COOCH₃), 3.52 (d, 214, CH₂, propyl-H), 5.49 (s, 1H, CH). ¹³C NMR (CDCl₃, 6 ppm): 15.70, 16.50, 17.50, 17.62, 18.80, 21.40, 23.70, 24.00, 25.00, 28.20, 28.80, 28.95, 31.10, 33.70, 37.40, 37.50, 39.20, 39.40, 39.50, 39.60, 40.10, 42.60, 43.80, 45.10, 48.10, 48.25, 52.68, 53.60, 55.90, 61.10, 78.20, 125.70, 139.30, 172.70 (34 C). MS (EI): m/z 526 (100%) [M⁺]. Anal. Calcd. for C₃₄H₅₄O₄ (526.79): Calcd. C, 77.52; H, 10.33. Found C, 77.42; H, 10.28.

### Example 2

### Synthesis of methyl-3-β-[2-hydroxy-3-(isopropyl- or isobutylamino)propoxy]-urs-12-en-28-oate (3a,b)

A solution of methyl ursolate (2) (5.26 g, 10 mmol) and the corresponding amine, namely, isopropylamine or isobutylamine (12 mmol) in dioxane (100 mL) was refluxed for 7 hrs. The reaction mixture was evaporated to dryness and washed with HCl, followed with water, and finally crystallized from methanol to give the corresponding products 3a, 3b, respectively.

Methyl-3-β [2-hydroxy-3-(isopropylamino)propoxy]-urs-12-en-28-oate (3a). Yield 78%, mp. 268-270° C., [α]_{D}²⁵=+108 (c 1, CHCl₃); IR (KBr, cm⁻¹): 2961 (CH, aliphatic), 1748 (ester), 1631 (C═C), 1275 (ether). ¹H NMR (400 MHz, CDCl₃, δ in ppm): 0.87 (d, 1H, CH), 0.91 (s, 3H, CH₃), 0.96 (d, 3H, CH₃), 0.98 (d, H, 1H, CH), 1.00 (s, 3H, CH₃), 1.03 (d, 3H, CH₃), 1.05 (m, 1H, CH), 1.08 (s, 3H, CH₃), 1.11 (d, 6H, 2CH₃), 1.21 (m, 1H, CH), 1.24 (s, 3H, CH₃), 1.28 (s, 3H, CH₃), 1.35 (m, 1H, CH), 1.38 (m, 1H, CH), 1.42 (m, 1H, CH), 1.45 (m, 1H, CH), 1.48 (m, 1H, CH), 1.52 (d, H, 1H, CH), 1.55 (m, 1H, CH), 1.58 (m, 1H, CH), 1.65 (s, 1H, CH), 1.80 (dd, 2H, CH₂), 1.90 (m, 2H, CH₂), 1.94 (m, 1H, CH), 1.97 (m, 1H, CH), 2.00 (m, 1H, CH), 2.13 (t, 1H, CH), 2.30 (t, 1H, CH), 2.35 (d, 2H, NCH₂), 2.61 (d, 1H, CH), 2.88 (m, 1H, NCH), 3.07 (s, 1H, NH), 3.44 (dd, 1H, CH), 3.49 (s, 3H, COOCH₃), 3.52 (d, 2H, CH₂), 3.72 (d, 1H, OH), 4.04 (m, 1H, CH), 5.48 (s, 1H, CH). ¹³C NMR (CDCl₃, δ in ppm): 15.60, 16.90, 17.30, 18.60, 21.20, 23.70, 24.10, 25.10, 28.30, 28.50, 28.80, 31.20, 33.70, 37.40, 37.48, 39.30, 39.50, 39.60, 39.70, 40.10, 42.40, 45.10, 45.16, 48.10, 48.20, 48.25, 52.80, 53.60, 55.70, 61.10, 62.80, 78.40, 125.70, 139.50, 172.40 (37 C). MS (EI): m/z 585 (100%) [M⁺]. Anal. Calcd for C₃₇H₆₃NO₄ (585.90): Calcd C, 75.85; H, 10.84; N, 2.39. Found C, 75.78; H, 10.80; N, 2.35.

Methyl-3-(3 [2-hydroxy-3-(isobutylamino)propoxy]-urs-12-en-28-oate (3b) Yield 70%, mp. 290-292° C., [α]_{D}²⁵=+149 (c 1, CHCl3); IR (KBr, cm-1): 2960 (CH, aliphatic), 1747 (ester), 1629 (C═C), 1275 (ether). ¹H NMR (400 MHz, CDCl₃, δ in ppm): 0.88 (d, 1H, CH), 0.91 (s, 3H, CH₃), 0.95 (d, 3H, CH₃), 0.99 (d, H, 1H, CH), 1.02 (s, 3H, CH₃), 1.05 (d, 3H, CH₃), 1.08 (m, 1H, CH), 1.11 (s, 3H, CH₃), 1.15 (d, 6H, 2CH₃), 1.20 (m, H, CH), 1.23 (s, 3H, CH₃), 1.29 (s, 3H, CH₃), 1.36 (m, 1H, CH), 1.37 (m, 1H, CH), 1.40 (m, 1H, CH), 1.46 (m, 1H, CH), 1.49 (m, 1H, CH), 1.52 (d, H, 1H, CH), 1.54 (m, 1H, CH), 1.60 (m, 1H, CH), 1.65 (s, 1H, CH), 1.80 (dd, 2H, CH₂), 1.91 (m, 2H, CH₂), 1.94 (m, 1H, CH), 1.98 (m, 1H, CH), 2.01 (m, 1H, CH), 2.12 (m, 1H, CH), 2.15 (t, 1H, CH), 2.31 (t, 1H, CH), 2.38 (d, 2H, NCH₂), 2.54 (d, 2H, NCH₂), 2.65 (d, 1H, CH), 2.86 (m, 1H, NCH), 3.46 (dd, 1H, CH), 3.48 (s, 3H, COOCH₃), 3.54 (d, 2H, CH₂), 3.75 (d, 1H, OH), 4.10 (m, 1H, CH), 5.46 (s, 1H, CH). ¹³C NMR (CDCl₃, δ in ppm): 15.70, 16.70, 17.40, 17.60, 18.90, 21.30, 23.40, 23.80, 24.20, 25.20, 28.30, 28.42, 28.70, 31.30, 33.80, 37.30, 37.50, 39.30, 39.50, 39.80, 39.92, 40.20, 42.40, 45.10, 45.18, 45.25, 48.30, 48.36, 48.50, 52.90, 53.80, 55.80, 61.30, 62.90, 78.40, 125.60, 139.50, 172.50 (38 C). MS (EI): m/z 600 (100%) [M⁺]. Anal. Calcd for C₃₈H₆₅NO₄ (599.92): Calcd 76.08; H, 10.92; N, 2.33. Found 76.00; H, 10.88; N, 2.28.

### Example 3

### Beta Blocker Activities

To test beta adrenergic receptor blocking effects of compounds 3a and 3b, their actions on the inotropic effects of the beta receptor agonist isoprenaline of the guinea pig left atrium were studied.

Male guinea-pigs weighing 300±400 g were killed by a blow on the head. The heart was quickly removed and placed in oxygenated Krebs-bicarbonate solution. The left atrium was carefully dissected and placed between bipolar platinum electrodes. The base of the atrium was tied to one of the electrodes and the tip to an isometric transducer connected to an amplifier and recorder (Cibertec, Madrid, Spain), with 1 g baseline tension. The preparation was placed in a 10-ml glass bath at 328 C, in Krebs-bicarbonate solution of the following composition (in mM): NaCl 119, KCl 4.7, MgSO4 1.2, KPO4H2 1.2, CaCl₂ 1.8, NaHCO₃ 25, glucose 11, pH 7.4. An initial 30-min period of electrical drive (4 V, 1 ms, 1 Hz) allowed the stabilization of the basal contraction. Thereafter, 361078 M isoprenaline (ISO) was added to the bath to enhance the contraction and increasing concentrations of each drug were added in order to study their blocking effects.

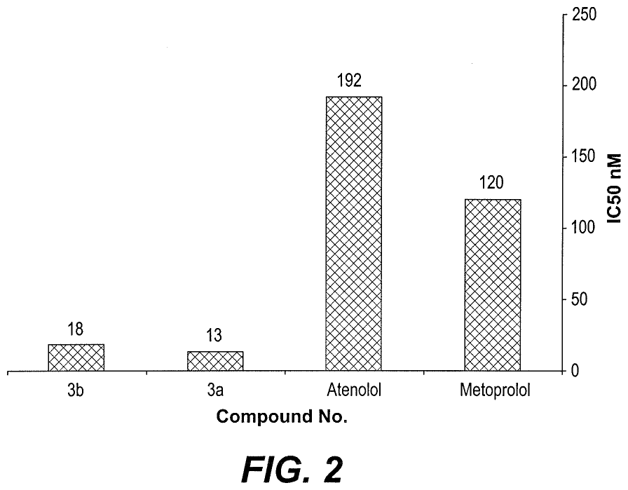

After an initial equilibration period, isoprenaline addition at a concentration of 3×10⁻⁸ produced a marked enhancement of atrial contractions. This concentration was selected because it produced a submaximal and measurable inotropic effect that was reproducible after 7⁻¹° additions of isoprenaline, given at 30-min intervals. In ten atria, the net increase in the force of contraction induced by isoprenaline amounted to 11+1.3 mN. Concentration-response curves for the inhibition of isoprenaline-induced inotropism were obtained by intercalating increasing concentrations of each compound between two isoprenaline additions. Each individual preparation served to provide a full concentration-response curve for a given compound, and from it IC₅₀ was calculated. As shown in Table 1 and FIG. 2, Compounds 3a and 3b blocked the isoprenaline inotropic response almost fully, as demonstrated by their IC₅₀. Both compounds 3a and 3b were more potent and active than Metoprolol and Atenolol. Compound 3a was more active than compound 3b.

TABLE 1
IC₅₀ of the beta blocker activities for compounds 3a and 3b.

| Compound | IC₅₀ (nM) |
|---|---|
| Metoprolol | 120 ± 1.89 |
| Atenolol | 192 ± 2.34 |
| 3a | 13 ± 0.28 |
| 3b | 18 ± 0.34 |

Values are mean ± S.E.M, n = 6 in each group; Statistical analysis by one way ANOVA followed by Dunnet test using Graphpad Instat software (P < 0.05).

### Example 4

### Molecular Docking Studies

Molecular Docking studies mainly aim to clarify the possible conformation of ligands in the binding pocket of a protein and to determine the affinity between the ligand and the protein. The minimum energy of interaction is represented by different scoring functions. In this study, MolDock score and hydrogen bonding energy were used. The crystal structure of β1-Adrenergic receptor (PDB 2vt4) was provided from Brookhaven protein data bank (PDB; http://www.rcsb.org/pdb) and loaded to Molegro Virtual Docker (MVD 2013.6.0.0 [win32]) program.

The protein preparation was carried out in two steps, preparation and refinement. After ensuring chemical correctness, the non-bonded oxygen atoms of water, present in the crystal structure, were removed and hydrogens were added where hydrogen atoms were missing. Formal charges and potential steric clashes via protein minimization were assigned. ChemBio3D Ultra 16 was used to draw the 3D structures of different ligands. Ligands were further pre-optimized using a free version of Marvinsketch 4.1.13 from Chemaxon Ltd with MM force field and saved in Tripos mol2 file format. Optimization of ligand conformations were converged to a gradient RMSD below 0.05 kJ/mol or continued to a maximum of 1,000 iterations, at which point there were negligible changes in RMSD gradients. The binding sites are defined by constraints at XYZ 10.82, 19.86, 22.41. The search began with a rough positioning and scoring phase that significantly narrowed the search space and reduced the number of poses to be further considered to a few hundred. In the following stage, the selected poses were minimized and in the final stage, the 5-10 lowest-energy poses obtained in this fashion were selected.

Docking of the tested compounds into the active site of β1-Adrenergic receptor (PDB 2vt4) structure give the MolDock scores and hydrogen bonding energies shown in Table 2 which indicate different affinities of the compounds with the receptor.

TABLE 2
Molecular docking results of compounds 3a and 3b

| compound | MolDock scores | Hydrogen bonding energy |
|---|---|---|
| Metoprolol | −108.02 | −3.71 |
| Atenolol | −116.64 | −3.79 |
| 3a | −99.96 | −2.50 |
| 3b | −93.66 | −3.94 |

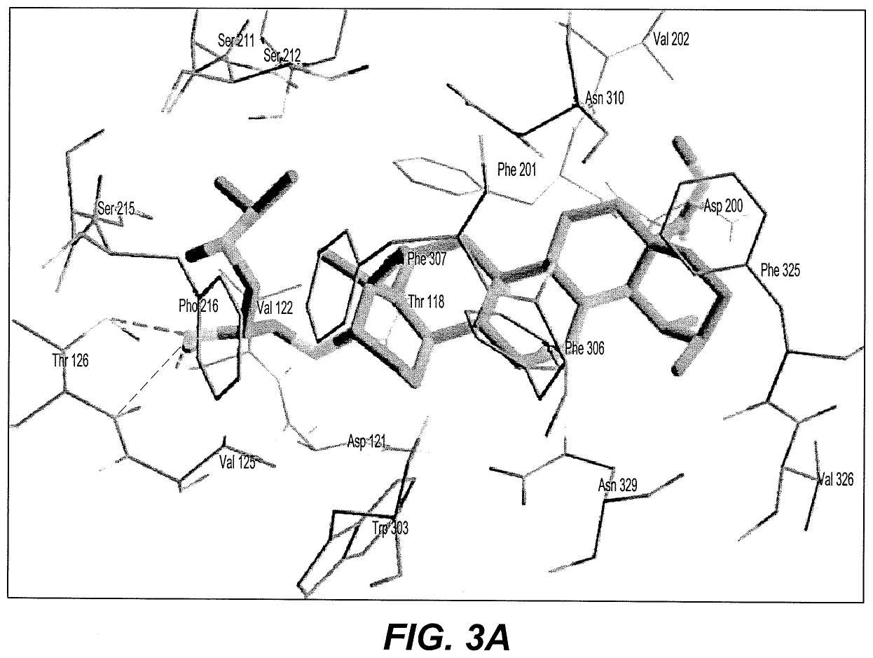

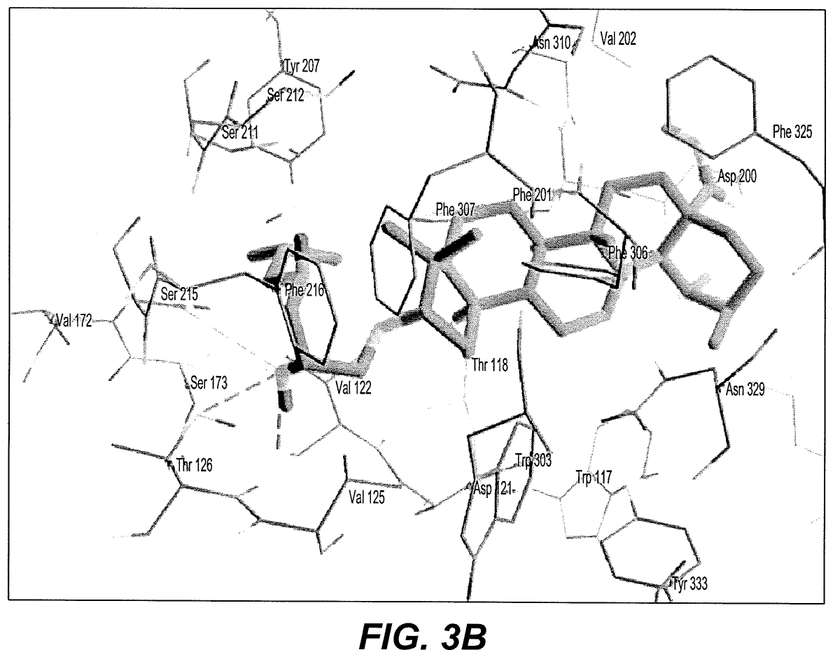

As shown in FIGS. 3A and 3B, Compound 3a makes three hydrogen bonds with the active site amino acids; OH_{side chain} makes two hydrogen bonds with NH and OH of Thr126, and COₑₛₜₑᵣ with Phe201. The ursolic acid rings of 3a make hydrophobic interactions with Phe201, Tyr207, Phe306 and Asn329 residues. On the other hand compound 3b interact with the active site amino acids with three hydrogen bonds between OH_{side chain} group with Thr126, Va1122 and Ser215 residues. In addition to hydrogen bonds there are hydrophobic interaction between the ursolic acid moiety and Trp117, Phe201, Trp303, Phe306, Phe207, Phe325 and Tyr333 residues.

It is to be understood that the beta-adrenergic blocking compounds as potent selective β1-blockers is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A beta-adrenergic blocking compound for treating hypertension, comprising a compound having the formula: 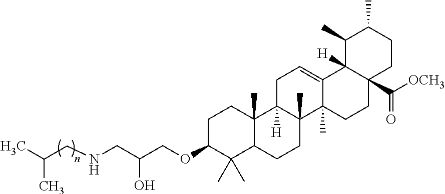
wherein n is 0 or 1.

2. The beta-adrenergic blocking compound according to claim 1, wherein n is 0.

3. The beta-adrenergic blocking compound according to claim 1, wherein n is 1.

4. A pharmaceutical composition, comprising the beta-adrenergic blocking compound according to claim 1 and a pharmaceutically acceptable carrier.

5. The pharmaceutical composition according to claim 4, wherein n is 0.

6. The pharmaceutical composition according to claim 4,
wherein n is 1.

7. A method for treating a patient suffering from a disease, comprising the step of administering to a patient a therapeutically effective amount of the pharmaceutical composition of claim 4, wherein the disease is comprises hypertension.
